(19) United States
(12) Patent Application Publication
(10) Pub. No.: US 2018/0195033 A1
(43) Pub. Date: Jul. 12, 2018

(54) CELL CULTURE DEVICE

(71) Applicants: PARIS SCIENCES ET LETTRES - QUARTIER LATIN, Paris (FR); UNIVERSITE PIERRE ET MARIE CURIE - PARIS 6 (UPMC), Paris (FR); CENTRE NATIONAL DE LA RECHERCHE SCIENTIFIQUE, Paris (FR)

(72) Inventors: Yong CHEN, Paris (FR); Yadong TANG, Bourg-la-Reine (FR); Li WANG, Paris (FR); Jian SHI, Meudon (FR)

(21) Appl. No.: 15/741,803

(22) PCT Filed: Jul. 8, 2016
(86) PCT No.: PCT/EP2016/066366

§ 371 Date: Jan. 4, 2018

(30) Foreign Application Priority Data
Jul. 8, 2015 (EP) 15175960.2

(51) Int. Cl. C12M 1/12; C12N 5/074; C12N 5/077; C12N 5/0793; C12M 1/32; C12M 1/00

(52) U.S. Cl. CPC C12M 25/02; C12N 5/0696; C12N 5/0657; C12N 2506/45; C12M 23/12; C12M 23/20; C12N 5/0619

(57) ABSTRACT

Disclosed is a cell culture device including a mesh including or made of a biocompatible polymer; and a top grid including or made of a biocompatible polymer, laying over the mesh; wherein the mesh is a monolayer of cross-linked nanofibers and has a specific surface ranging from 20% to 40%; the top grid includes a single grid and an array of openings separated by partitions having a width; each opening of the top grid has the same geometric configuration; and the top grid includes a border surrounding the openings, the border having a width at least two times greater than the width of the partitions. Also disclosed is a method for manufacturing the cell culture device, a method of cell growth or differentiation and a cell culture system.

### FIELD OF INVENTION

The present invention pertains to the field of tissue or cell culture device. Especially, the invention relates to a cell culture device comprising a layer of nanofibers. The present invention also relates to a method for manufacturing said cell culture device, a method of cell growth and cell differentiation and a cell culture system.

### BACKGROUND OF INVENTION

Tissue or cell culture and cell differentiation are complex processes which require mimicking the in-vivo physiological conditions. With conventional tissue or cell culture and differentiation technics, cells rest on a flat support, such as glass substrates or plastic substrates, without underneath diffusion of cell culture medium: only a part of the surface of the cells are in contact with the surrounding culture medium. Even if the conventional methods enable culture of some cell populations, pluripotent stem cells, such as embryonic stem cells and induced pluripotent stem cells (iPSC) require in-vitro conditions mimicking much more adequately in vivo conditions, wherein the whole surface of each cell is in contact with the extracellular matrix.

Among new cell culture devices, the use of nanofibers has been well studied in recent years. For instance, International patent application WO2015/007797 describes a three-dimensional scaffold for cell culture. The bio compatible scaffold is made of a three-dimensional nanofibrous scaffold covered with micro-tissues, such as an alginate hydrogel comprising living cells. With such device, the tissue regeneration proceeds in depth, up to the core of the scaffold.

U.S. patent application 2014/0207248 discloses a multi-scale fibrous scaffold comprising nanofibers and microfibers providing a three-dimensional environment for cell growth. The microfibers provide mechanical support and larges pores for cell infiltration while the nanofibers provide surfaces for cell adhesion.

International patent application WO2013/007224 also describes a cell culture substrate comprising a nanofibers layer deposited on a bearing stratum formed by a reticule. The nanofibers layer, formed from a biologically compatible polymer such as gelatin, polycaprolactone or polyamide, fills up and covers the pores of the bearing stratum. The said bearing stratum provided the substrate with required mechanical properties as the nanofibers layer, as such, exhibits insufficient mechanical strength and rolls up and shrinks after wetting. To prevent the nanofiber layer from mechanical damages, the said nanofiber layer may be covered with a polyethylene foil.

WO2015/007797, U.S. 2014/0207248 and WO2013/007224 aims at providing nanofibers scaffold suitable for cell differentiation and cell growth. However, they disclose three-dimensional scaffold of nanofibers. WO2015/007797 discloses indeed a scaffold having a thickness above 50 μm, advantageously up to 50 mm; and U.S. 2014/0207248 and WO2013/007224 describe the manufacturing of the nanofibers layers by electrospinning: said manufacturing process, as such, creates a three-dimensional structure. Within such three-dimensional exogenous environment, the cells are not fully immerged within the cell culture medium. Such requirement is necessary, especially for cell fate regulation of pluripotent stem cells. Indeed, with the culture devices of the prior art, pluripotent stem cells show important chromosomal abnormalities and high tumorigenic risk. It is therefore an object of the invention to provide a cell culture device mimicking in vivo conditions with enhanced permeability, decreased exogenous contact and increased contact area with the cell culture medium.

U.S. 2014/0295553 discloses a cell culture device comprising a crosslinked hydrogel layer bonded to a micro pattern plate.

In U.S. 2014/0295553, the culture device is made of a flat layer of hydrogel which cannot provide optimal cell culture condition, because of lack of 3D micro-environment. Even though the culture medium may be diffused cross the gel layer, the exchange efficiency between cells and the medium is always limited.

A second object of the invention is to provide a cell culture device allowing homogeneous seeding and growing of cell populations. This invention provides a surprisingly effective and original solution to both first and second object of the invention, though the use of a grid with openings covering a layer of nanofibers thereby allowing homogeneous seeding and growing within each openings; as well as the growth of different cell populations within a single device.

Furthermore, layers superimposition, as discloses within WO2015/007797 and U.S. 2014/0207248 strongly limits the cell imaging. A third object of the invention is therefore to provide a cell culture device wherein the cell growth and differentiation may be monitor by means of optical microscopy without damaging to the cell culture device.

### SUMMARY

To that end the cell culture device of the invention comprises a monolayer of cross-linked nanofibers which exhibits a mesh having holes of a size slightly smaller than the size of the cells to be cultured. Consequently, the cells merely rest on the nanofibers monolayer, acting as a net. The cells cover the holes and are in contact with the nanofibers, but along the border of the holes only; thereby optimizing the surface of the cells in contact with the cell culture medium: according to the invention, the cells are indeed in contact with the cell culture medium on their whole surface except on the border of the holes.

According to the Applicant, such new nanofibers monolayer supports the mimic in-vivo organization of the extracellular matrix and takes into account the hydrodynamic properties of the in-vivo cellular environment; thereby allowing significant increase of the proliferation rate and precise tuning of the shape of the iPSC colonies. The cell culture device of the invention also comprises a grid with openings on the top of the nanofibers monolayer allowing deposition of cells within each opening.

The present invention thus relates to an easy to handle and versatile cell culture device comprising a mesh comprising or made of a biocompatible polymer; and a top grid comprising or made of a biocompatible polymer, laying over the said mesh; wherein the mesh is a monolayer of cross-linked nanofibers and has a specific surface ranging from 20% to 40%; the top grid comprises a single grid and an array of openings separated by partitions having a width; each opening of the top grid has the same geometric configuration; and the top grid comprises a border surrounding the openings, the said border having a width at least two times greater than the width of the said partitions.

According to one embodiment, the said geometric configuration of the openings is a polygon, preferably a regular polygon such as an equilateral triangle or a regular hexagon.

According to one embodiment, each partition of the top grid separating the openings has the same cross-section, preferably a square cross-section, with a width ranging from about 5 to about 500 μm. According to one embodiment, the top grid has a border thicker than the partition of the top grid. According to one embodiment, the cell culture device further comprises a binding agent between the top grid and the mesh, said binding agent being preferably gold. According to one embodiment, more than 50% of the pores of the said mesh have an area ranging from about 0.01 to about 20 μm2. According to one embodiment, the openings of the top grid have dimensions ranging from about 200 to about 1000 μm. According to one embodiment, the nanofibers of the mesh comprise or are made of an hydrogel, preferably gelatin; or a doped hydrogel preferably gelatin doped with carbon nanotubes. According to one embodiment, the top grid comprises or is made of an hydrogel, preferably poly (ethylene glycol) or poly (ethylene glycol) diacrylate.

According to one embodiment, the cell culture device further comprises stem cells within the openings of the said top grid.

The present invention also relates to a cell culture system comprising at least one cell culture device according to the present invention; and a culture medium.

According to one embodiment, the mesh and the top grid of the at least one cell culture device comprise or are made of hydrogels such that the at least one cell culture device may be suspended within the cell culture medium. According to one embodiment, the cell culture system further comprises an inlet port, an outlet port and a microchannel, wherein the said culture medium and the said at least one cell culture device are comprised within the microchannel.

The present invention also relates to a method for manufacturing a cell culture device according to the invention, comprising:
manufacturing a grid made from biocompatible polymer by soft-lithography;
optionally depositing a binding agent on the grid by sputtering;
depositing a nanofibers layer on the grid by electrospinning;
cross-linking the said nanofibers.

The present invention also relates to a method of stem cell growth or differentiation comprising the following steps:
providing a cell culture device or a cell culture system according to the present invention;
optionally coating the cell culture device with glycoprotein such as vitronectin or fibronectin;
seeding at least one type of stem cells within the openings of the cell culture device in a medium optionally containing ROCK inhibitor; and
optionally, removing ROCK inhibitor.

### DEFINITIONS

In the present invention, the following terms have the following meanings:

The term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent, preferably of 5 percent, more preferably 1 percent.

“Crosslinker” refers to polyfunctional molecules capable to chemically react with specific functional groups (primary amines, sulfhydryls, etc) and bond them together.

“Culture medium” refers to a liquid or gelatinous substance in which microorganisms, cells or tissues are cultivated.

“Grid” refers to a three-dimensional architecture containing openings described by their regular geometric configuration. The grid is defined as its openings are at a microscopic scale.

“Hydrogel” refers to a non-fluid polymer network that is expanded throughout its whole volume by water.

“Monolayer” refers to a layer having one dimension (height or thickness) smaller than the other dimension(s) (length and width; or diameter). In the sense of the present invention, the smallest dimension (height or thickness) is smaller than the other dimension(s) (length and width; or diameter) by a factor of at least 5, 10, 15 or 20.

“Nanofiber” refers to a fiber whose diameter is less than 1 μm.

“Opening” refers to an aperture of the whole thickness of a material from one face to the opposite face.

“Porosity” refers to a quantity in percentage of openings compared to the whole surface in a material. Within the present invention, the term porosity refers to a surface porosity.

“Specific surface” refers to the ratio between the projection area of the nanofibers over the total mesh surface.

“Suspended culture device” refers to a culture device maintained in liquid between the surface and the bottom. Herein suspended means that once the device has been positioned in the liquid, the device does not sink nor resurface.

“Versatile” refers to a material whereon one or more cell lines is realized.

### DETAILED DESCRIPTION

The subject matter of the present invention is an easy to handle and versatile cell culture device, comprising:
a mesh comprising or made of a biocompatible polymer; and
a top grid comprising or made of a biocompatibie polymer, laying over the said mesh;

wherein
the mesh is a monolayer of cross-linked nanofibers and has a specific surface ranging from 20% to 40%;
the top grid comprises a single grid and an array of openings separated by partitions having a width;
the openings of the top grid have the same geometric configuration; and
the top grid comprises a border surrounding the openings, the said border having a width at least two times greater than the width of the said partitions.

As depicted in FIG. 1, the cell culture device 1 comprises a mesh 11 and a top grid 12 and the top grid comprises a border 121.

The mesh 11 according to the invention is made of a biocompatible material. Said biocompatible material may either be synthetic or natural. According to one embodiment, the nanofibers are made of hydrogel, preferably gelatin, or a doped hydrogel, preferably gelatin doped with carbon nanotubes. Doped hydrogel, such as gelatin doped with carbon nanotubes enhances the conductivity and the mechanical properties of the mesh 11.

Said mesh 11 is a monolayer of cross-linked nanofibers. According to one embodiment, the mesh has a thickness, in the z direction, ranging from about 20 to about 2500 nm, preferably from about 50 to about 1500 nm and more preferably from about 100 to about 500 nm. According to one embodiment, the mesh has a thickness, in the z direction, lower than 1 μm.

According to one embodiment, the device comprises only nanofibers and does not comprise microfibers.

According to one embodiment, the nanofibers have a diameter ranging from about 20 to about 1500 nm, preferably from about 100 to about 500 nm.

The specific surface of the mesh 11 is represented in FIG. 2c). According to one embodiment, the specific surface of the mesh 11 is not more than 40%, 35%, 30% or 25%. According to one embodiment, the specific surface of the mesh 11 is not less than 20%, 15% or 10%. According to one embodiment, the specific surface of the mesh 11 is ranging from 20% to 40%. According to the Applicant, specific surface below 40% allows permeability, high transparency and enough support for cells culture on it. Specific surface higher than 40% prevents optimal underneath circulation when the cells are deposited onto the mesh 11 while specific surface lower than 20% does not provide sufficient support for cells culture.

According to one embodiment, the mesh comprises apertures. According to one embodiment, more than 50% of the apertures have an area ranging from about 0.01 μm² to about 20 μm² and preferably to about 5 μm².

According to one embodiment, the porosity of the said mesh 11 in the plane perpendicular to the smallest dimension (also referred to as in-plane or x-y plane) is not less than 60%.

According to one embodiment, the porosity of the mesh 11 is represented in FIG. 2c).

According to one embodiment, the porosity of the mesh 11 is not less than 50%, 55%, 60%, 65%, 70% or 75%. According to one embodiment, the porosity of the mesh 11 is not more than 80%, 85% or 90%. According to one embodiment, the porosity of the mesh 11 is ranging from 60% to 80%. According to the Applicant, porosity above 60% allows permeability, high transparency and enough support for cells culture on it. Porosity lower than 60% prevents optimal underneath circulation when the cells are deposited onto the mesh 11 while porosity higher than 80% does not provide sufficient support for cells culture.

According to one embodiment, more than 50% of the pores have an area ranging from about 0.01 μm² to about 20 μm² and preferably to about 5 μm².

The top grid 12 according to the invention is made of a biocompatible material. Said biocompatible material may either be synthetic or natural. According to one embodiment, the said top grid 12 comprises or is made of an hydrogel, preferably poly (ethylene glycol) or poly (ethylene glycol) diacrylate.

The said top grid 12 comprises a single grid and an array of openings separated by partitions. According to one embodiment, the partitions have a width in the x-y plane ranging from about 5 to about 500 μm, preferably from about 20 μm to about 100 μm, more preferably about 50 μm.

According to one embodiment, the top grid 12 has a thickness in the z axis ranging from about 5 to about 500 μm, preferably from about 40 μm to about 80 μm, more preferably about 50 μm.

According to one embodiment, each partition of the said top grid 12 has the same cross-section preferably a square cross-section.

According to one embodiment, the said top grid 12 can take any form, preferably a disc.

The said openings of the top grid 12 have the same geometric configuration. According to one embodiment, the said geometric configurations of the openings are polygons, preferably regular polygons such as an equilateral triangle or a regular hexagon (as shown in FIG. 2a). According to one embodiment, the said openings have dimensions from about 200 to about 1000 μm.

The said top grid 12 comprises a border 121 surrounding the array of openings and having a width in the x-y plane at least two times bigger than the width of the said partitions. The said feature enables easy handling of the cell culture device. According to one embodiment, the said border 121 has a width in the x-y plane at least 2, 4, 5, 10, 15, 20, 50 times bigger than the width of the said partitions.

According to one embodiment, the border 121 has a thickness in the z axis ranging from about 10 to about 5000 μm, preferably from about 50 μm to about 500 μm, more preferably about 100 μm.

According to one embodiment, the border 121 has the same thickness in the z direction than the grid 12. According to one embodiment, the border 121 has a thickness in the z axis ranging from 2 to 50 times thicker in the z axis than the top grid 12. According to one embodiment, the border 121 has a thickness in the z axis 2, 3, 4, 5, 10, 15, 20 or 50 times thicker in the z axis than the top grid 12.

According to one embodiment, the said border 121 has a thickness in the z axis ranging, an inner diameter in the x-y plane ranging from about 2 mm to about 50 mm, preferably from about 5 mm to about 20 mm, more preferably about 9 mm and an outer diameter in the x-y plane ranging from about 5 mm to about 60 mm, preferably from about 7 mm to about 25 mm, more preferably about 13 mm.

According to one embodiment, the said border 121 is made of the same material as the top grid 12. According to one alternative embodiment, the said border 121 is made of a different material as the top grid 12. According to one embodiment, the said border 121 comprises or is made of an hydrogel, preferably poly (ethylene glycol) or poly (ethylene glycol) diacrylate.

According to one embodiment, the top grid 12 is fixed to the nanofibers mesh 11 by electrostatic interactions. According to one embodiment and as schematically represented in FIG. 3, the top grid 12 is fixed to the nanofibers mesh 11 by a binding agent 13, preferably gold. According to one embodiment, the binding agent 13 has a thickness in the z axis of about 10 nm.

According to one embodiment, as depicted in FIG. 3, the top grid 12 comprises a border 121 surrounding the openings, the said border having a high at least two times higher than the high of the said partitions.

According to one embodiment, stem cells, preferably pluripotent stem cells (PSC), such as induced PSC (iPSC) are located within the openings of the said top grid 12.

According to one embodiment, the culture device 1 is covered or coated with glycoprotein such as vitronectin or fibronectin to promote the adhesion of stem cells on the mesh 11, preferably adhesion of PSC on the mesh, more preferably adhesion of iPSC on the mesh.

According to one embodiment, at least one cell culture device 1 as described before may be coupled to a culture medium to form a cell culture system. According to one embodiment and as shown in FIG. 4, the mesh 11 and the top grid 12 of the said at least one cell culture device 1 comprise or are made of hydrogels such that the said at least one cell culture device 1 may be suspended within the cell culture medium 3 without external support. According to one embodiment and as shown in FIG. 5, the said cell culture system comprises an inlet port, an outlet port and a microchannel 5, wherein the said culture medium 3 and the said at least one cell culture device 1 are comprised within the microchannel.

According to one embodiment, depicted in FIG. 6, the mesh 11, made of gelatin nanofibers, is fixed to the top grid 12, made of PEGDA (poly(ethylene glycol) diacrylate), by a binding agent 13, gold, to permit culturing cells 2 between the partitions.

According to another aspect, the invention relates to a method for producing the culture device 1 as described above, comprising:
manufacturing a grid 12 made from hydrogel by soft-lithography;
optionally depositing a binding agent 13 on the grid 12 by sputtering;
depositing a nanofibers layer 11 on the grid 12 by electrospinning; and
cross-linking the said nanofibers.

With regard to the first step of the method above, the grid 12 is made from hydrogel, more preferably from poly (ethylene glycol) or poly (ethylene glycol) diacrylate by soft-lithography. According to one embodiment, an hydrogel solution, more preferably a PEGDA solution fills a stamp partly made of silicon, more preferably from polydimethylsiloxane (PDMS)-glass assembly. The hydrogel solution is then exposed to UV. According to one embodiment, the said border 121 is prepared in a similar manner.

The optional second step of the method above consists of sputtering a binding agent 13, such as gold, on either one of the x-y surface. According to one embodiment, the top grid 12 has a border 121 on one x-y surface and the gold layer is sputtered on the other x-y surface of the said top grid 12. According to one embodiment, the top grid 12 does not have a border 121 and the binding agent 13 is sputtered on any surface of the x-y plane of the said top grid 12. According to one embodiment, the thickness of the binding agent 13 in the z direction is around 10 nm.

The third step of the method above consists of depositing a nanofibers layer 11, preferably made of an hydrogel and preferably of gelatin, on the x-y surface of the top grid 12 by electrospinning. According to one embodiment, monolayer of nanofibers can have optimized specific surface or porosity and pores size by adapting electrospinning parameters and electrospinning time.

The last step of the method above consists of the fibers cross-linkage.

According to one embodiment, the nanofibers are cross-linked by means of a crosslinker.

According to one embodiment, the crosslinker is selected from a bi-component system comprising carbodiimide and succinimide, preferably EDC (1-Ethyl-3-(3-dimethylaminopropyl)carbodiimide) and NHS (N-Hydroxysuccinimide) for optimal biocompatibility.

According to one embodiment, the nanofibers are cross-linked by soaking their surface in a solvent. According to one embodiment, the said solvent is associated to an ethanol solution with EDC (1-Ethyl-3-(3-dimethylaminopropyl)carbodiimide) and NHS (N-Hydroxysuccinimide).

According to one embodiment, the nanofibers are not crosslinked by means of free radicals.

According to the Applicant, on the contrary to layers of nanofibers obtained by electrospinning of the prior art, the cross-linkage of the layer post-electrospinning have a slimming effect on the layer and allows to reach a thickness equivalent of the monolayer.

Advantageously, the process for crosslinking the nanofibers does not provide any radical entities. The crosslinked nanofibers do not comprise any radical so that the mesh of biocompatible polymer avoids damaging the cells. Advantageously, the process for crosslinking nanofibers is versatile. Indeed, the process of the invention allows crosslinking the nanofibers from any biocompatible polymer having both a carboxyl and hydroxyl group; especially, the process of the invention allows crosslinking the nanofibers from any biocompatible polymer without the need to modify the chemical structure of said polymer before crosslinking step. Advantageously, the process for crosslinking monolayer nanofiber allows forming a mesh structure made of connected strands of nanofibers.

The invention further provides cell culture device obtainable by the methods described herein.

The present invention also relates to a method of cell growth and cell differentiation comprising the following steps:
providing a cell culture device according to anyone of claims 1 to 10;
the device is sterilized under UV exposure;
optionally coating the cell culture device with a glycoprotein such as fibronectin or vitronectin; and
seeding at least one type of stem cells within the openings of the cell culture device in a medium optionally containing Rho-associated protein kinase (ROCK) inhibitor; and
optionally, removing ROCK inhibitor.

### BRIEF DESCRIPTION OF THE DRAWINGS

FIG. 1 is a schematic sectional view of the cell culture device 1 in the z axis according to one embodiment of the invention.

FIG. 2 is a multi-scale analysis by SEM images of the cell culture device 1 according to one embodiment of the invention in the x-y plane, zoom in from the top grid to the monolayer of nanofibers, 2a being a view of the top grid 12, 2b being a view of one opening of the top grid 12 and 2c being a view of the monolayer of nanofibers 11.

FIG. 3 is a sectional view of the cell culture device 1 in the z axis according to one embodiment of the invention with a binding agent between the monolayer of nanofibers 11 and the top grid 12.

FIG. 4 is a sectional view of the cell culture system 4 according to one embodiment of the invention comprising a cell culture device 1 suspended in a cell culture medium 3 for culturing cells 2.

FIG. 5 is a sectional view of the cell culture system 4 according to one embodiment of the invention comprising a cell culture device 1 suspended in a cell culture medium 3 for culturing cells 2, the cell culture medium being transferred through micro-channels 5.

FIG. 6 is a top view (A), a global sectional view (B) and a local sectional view (C) of the cell culture device 1 according to one embodiment of the invention, comprising a PEGDA grid 12 and border 121, a layer of binding agent gold 13 and a monolayer of gelatin nanofibers 11, wherein cells 2 are stuck on the monolayer of gelatin nanofibers 11.

FIG. 7 shows three scale SEM images (a: top grid scale, b: opening of the top grid scale and c: nanofibers layer) of three monolayers of gelatin nanofibers 11 depending on the electrospinning time (from 7 to 30 minutes) with significant changes of the specific surface of cross-linked nanofibers.

FIG. 8 are histograms of pore sizes of two types of monolayer of gelatin nanofibers 11 depending on the electrospinning time with most of the pores being from about 0 to about 20 μm² for 7 min electrospinning (a) and being from about 0 to about 5 μm² for 15 min electrospinning (b).

FIG. 9 are Bright Field (BF), fluorescence images of NIH-3T3 and HeLa cells: phalloidin for cytoskeleton F-actin and DAPI for nuclear, and merge images.

FIG. 10 shows the increase of the NIH-3T3 cell number versus culture time with a cell culture device according to one embodiment of the invention (black curve) and a normal culture dish (grey curve) for 72 h, presenting a significant difference of the cell proliferation rate between the two types of device.

FIG. 11 is a microphotograph of hiPSC colonies formed in the center of the opening of a PEGDA grid, showing homogenous distribution and dome-like morphology.

FIG. 12 are bright field photos, SEM images of hiPSC colonies and a histogram showing the distribution of the colonies diameters. The hiPSC colonies are formed in the center of openings of PEGDA grid, showing dome-like and disk-like morphologies which can be controlled by changing the during time of ROCK inhibitor treatment.

FIG. 13 are fluorescence and bright field images of a hiPSC colony formed in the center of an opening of a PEGDA grid, showing the most of hiPSC are alive.

FIG. 14 is a schematic diagram of cardiac differentiation of hiPSC on a PEGDA/monolayer of nanofibers mesh. hiPSCs colonies are formed in each of the openings of PEGDA grid after seeding the cells on the PEGDA grid and a ROCK inhibitor treatment. Cardiomyocytes are then obtained after adding cardiac differentiation induction factors.

FIG. 15 is a schematic diagram of cardiac differentiation sequence of hiPSCs on the cell culture device made of a PEGDA grid and a monolayer of nanofibers.

FIG. 16 is a fluorescence image of cardiomyocytes derived from hiPSCs in an opening of PEGDA grid (DAPI, α-actinin, cTnT2), indicating the formation of sarcomere structures.

FIG. 17 is a fluorescence image of motor neuron progenitors derived from hiPSCs in an opening of the PEGDA grid (DAPI, Olig2 and Tubulin), indicating a relatively high differentiation rate.

FIG. 18 is a top view photography of a cell culture device 1 according to one embodiment of the invention.

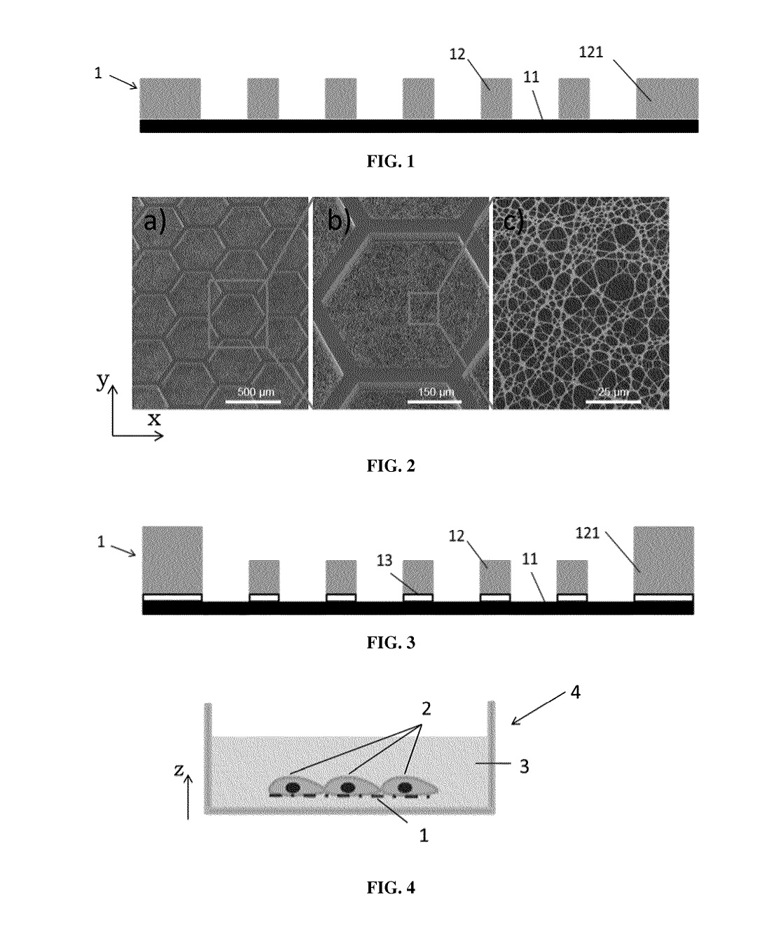

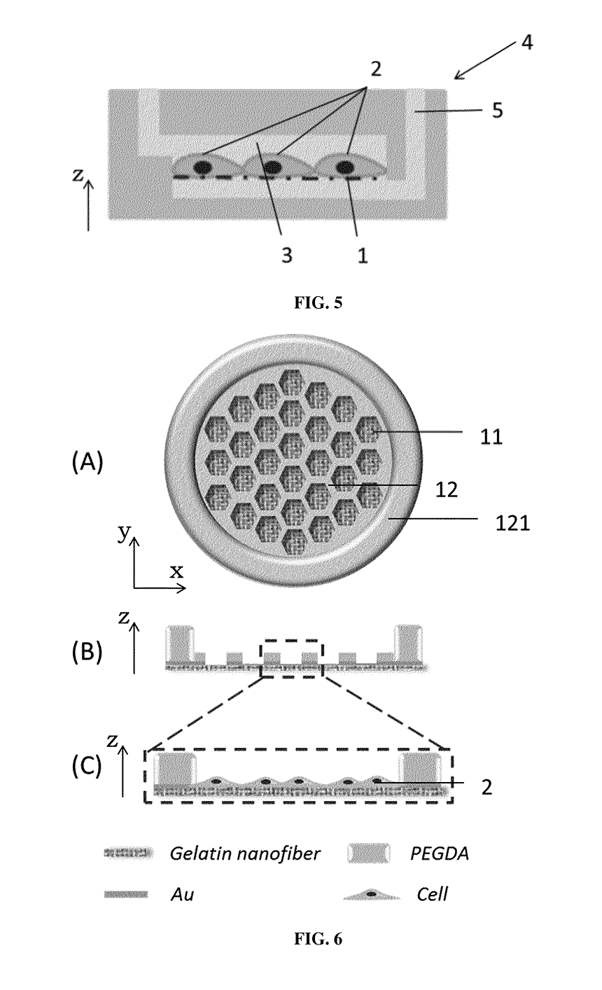

### REFERENCES

1—Cell culture device;

11—Mesh/monolayer of cross-linked nanofibers;

12—Top grid;

121—Border surrounding the top grid;

13—Binding agent;

2—Culturing cells;

21—hiPSC colonies;

3—Culture medium;

4—Cell culture system;

5—Microchannel.

### EXAMPLES

The following discussions present non-limiting examples of certain embodiments of the methods, devices and systems of the present invention. Persons having ordinary skill in the relevant arts and possession of the present disclosure may make numerous modifications and variations on these embodiments without departing from the spirit and scope of the invention.

The present invention is further illustrated by the following examples of processing the device or uses:

### Material and Methods

SEM Observation

Samples are fixed in PBS containing 4% formaldehyde for 30 minutes. Then, they are rinsed twice with PBS buffer, and immersed in 30% ethanol (in distilled water (DI)) for 30 minutes. Afterward, the samples are dehydrated in a graded series of ethanol with concentrations of 50%, 70%, 80%, 90%, 95%, and 100%, respectively, each for 10 min and dried with a nitrogen gas flow. Before observation, a 2 nm thick gold layer is deposited on the samples by sputtering. The observation is performed with a scanning electron microscope (Hitachi S-800) operated at 10 kV.

Immunofluorescence Staining and Observation

First, the dome-like hiPSC aggregates are fixed in 4% v/v paraformaldehyde at room temperature for 30 min, permeabilized with 0.5% v/v Triton X-100 in Dulbecco's Phosphate-Buffered Saline (DPBS) at 4° C. overnight and incubated with blocking solution containing 5% v/v normal goat serum, 5% v/v normal donkey serum, 3% v/v bovine serum albumin and 0.1% v/v Tween 20 in DPBS at 4° C. overnight. Cells are then incubated with primary antibodies, i.e., anti-OCT4 (2 μg mL-1), anti-NANOG (9.4 μg mL-1), anti-SOX17 (20 μg mL-1), anti-β-tubulin III (6 μg mL-1), or anti-alpha smooth muscle actin (2 μg mL-1) in 0.5 v/v % Triton X-100 in DPBS at 4° C. overnight. Following incubation with the primary antibody, cells are incubated with the appropriate secondary antibody, i.e., DyLight-649 anti-rabbit IgG (0.375 or 3 μg mL-1) or DyLight 488 anti-mouse IgG (1.5 μg mL-1), in blocking buffer at room temperature for 1 h. Finally, cell nuclei are stained with 300 nM 4′-6-diamidino-2-phenylindole (DAPI) at room temperature for 30 min.

The differentiated cardiomyocytes on monolayers of nanofibers are fixed with 4% paraformaldehyde (PFA) diluted in DPBS for 15 min. Then cells are treated with 0.2% Triton X-100 in DPBS for 1 h for permeabilization and then 1% bovine serum albumin (BSA) in DPBS is added overnight at 4° C. to block out non-specific bindings. Afterwards, cells are incubated with primary antibodies of Anti-α-Actinin (Sarcomere) antibody and anti-TnnT2 over night at 4° C. Cells are then washed with DPBS 3 times of 5 min. Then cells are immersed in secondary antibodies of donkey anti-mouse cy3 and donkey anti-goat cy5 for 1.5 h at room temperature in the dark. After washing, cells are stained with 100 nM DAPI for 15 min at room temperature and following with 3 times 5 min PBS rinsing. Finally sample is mounted with histology mounting medium (Sigma, Fluoroshield™, F6182).

Fluorescence images are obtained with an inverted optical microscope (Zeiss, Axiovert 200) equipped with a digital CCD camera (Evolution QEI).

Live/Dead Assay

Cell viability is studied by live/dead assay. Briefly, 2 μM of Calcein AM and 2 μM EthD-1 are respectively added on the monolayer of nanofibers with dome-like iPS cell clusters grow and dead cell staining. After 30 min incubation at 37° C. and 5% CO2, cells are analyzed with a fluorescence microscope, as described above. Cell viability is calculated by live cells number divided by total cells number.

### Example 1: Grid Mask Fabrication Process

A chromium mask of regular hexagonal network array is produced by a micro pattern generator (μPG 101, Heidelberg Instruments). The regular hexagonal have a hexagonal openings period of about 500 μm in the x-y plane and about 50 μm line width to further produce the partitions. The mask is then spin-coated on one of the surface in the x-y plane with a about 50 μm thick photoresist (AZ40XT, MicroChem) and backside exposed with UV light. After development, the mask with photoresist patterns was treated in a vapor of trimethylchlorosilane (TMCS) for anti-sticking surface treatment. A mixture of PDMS (polydimethylsiloxane) pre-polymer and cross-linker (RTV 615, GE silicone rubber) was prepared at ratio of 10:1 and then poured on the treated chromium mask. After curing at 80° C. for 2 h, the PDMS layer was peeled off and placed on a glass slide. Afterward, the PDMS-glass assembly was placed in a desiccator for degasing during 15 min.

### Example 2: PEGDA Grid Fabrication Process

A PEGDA solution mixed with 1 v/v % Irgacure 2959 (1-[4-(2-Hydroxyethoxy)-phenyl]-2-hydroxy-2-methyl-1-propane-1-one) was prepared. The said solution is poured in the PDMS openings on the glass slide by degasing induced micro-aspiration, followed by UV exposure at 9.1 mW/cm² for 30 s. The PDMS mould is peeled off when the PEGDA network is solidified. An about 100 μm thick PEGDA border (13 mm outer diameter and 9 mm inner diameter) is prepared in a similar manner.

### Example 3: Gelatin Nanofibers Mesh Electrospinning Process on a PEGDA Grid

A solution of 10 wt % gelatin powder (G2625, Sigma-Aldrich, France) is dissolved in a mixture of acetic acid, ethyl acetate and distilled water with a volume ratio of 21:14:10. The solution is prepared 16 h before electrospinning. One of the x-y surfaces of the PEGDA grid is sputtered with about 10 nm thick Au to enhance adhesion of gelatin nanofibers on the PEGDA grid. The PEGDA grid with Au layer is placed on a silicon wafer used as a collector. The gelatin solution is loaded in a syringe and was ejected to the said collector at a distance of about 10 cm by the use of a syringe pump (KD Scientific) at 0.2 ml/h pumping speed through a stainless steel 23-gauge needle.

The spinneret is connected to the anode of high potential power supply (TechDempaz, Japan) with bias voltage of 11 KV and the collector is connected to the cathode of the power supply. After electrospinning, the samples are dried in vacuum overnight to get rid of the remaining solvent. Afterward, the electrospun gelatin nanofibers are cross-linked by soaking the substrate in an ethanol solution with 0.2 M EDC (1-Ethyl-3-(3-dimethylaminopropyl)carbodiimide) and 0.2 M NHS (N-Hydroxysuccinimide) for 4 h.

After crosslinking, samples are rinsed with ethanol three times and dried in vacuum overnight to get rid of the remaining chemicals, resulting in a complex net of PEGDA honeycomb supported monolayer nanofibers.

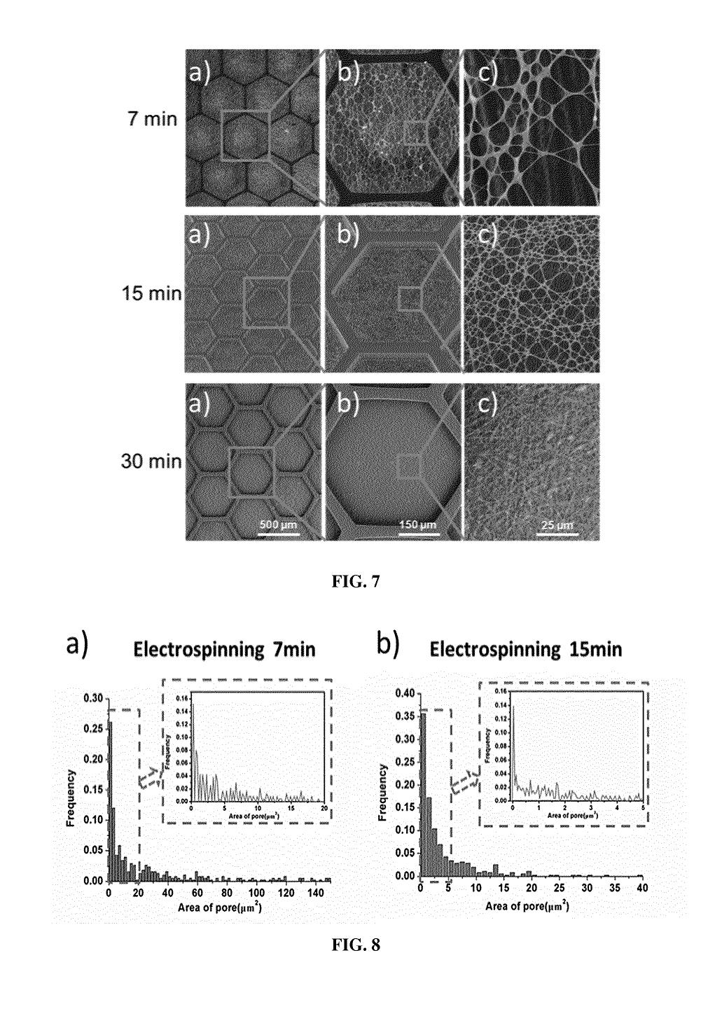

The diameter of gelatin nanofibers obtained by this process is in the range of 100-500 nm. To optimize the specific surface and openings of the nanofibers layer, different electrospinning time has been tested all other things being equal. Three different electrospinning times are studied: 7 min, 15 min and 30 min. The SEM image of the specific surface of the nanofibers monolayers are shown in FIG. 7. For both 7 min and 15 min of electrospinning, monolayer of nanofibers can be obtained but when electrospinning time is 30 min or more, monolayer of nanofibers cannot be obtained and there is almost no hole among fibers. Pores sizes are analyzed by ImageJ software from SEM images for electrospinning times of 7 min and 15 min, results are shown in FIG. 8. Porosity for 7 min and 15 min of electrospinning time are respectively about 79.8±0.8% and about 63.65±1.35%. Both of them are transparent enough for a final suspension culture system. For 7 min of electrospinning time, the area of most pores was from 0 to 20 μm², and some of them were even more than 100 μm², which is too large for cells stay at. The pores for 15 min spinning were mainly less than 5 μm² and almost no more than 20 μm², which can keep both high transparency and enough support for cells culture on it.

Example 4: HeLa and NIH 3T3 Off-Ground Cell Culture

Preparation of NIH 3T3 cells suspension: NIH 3T3 cells are cultured at 37° C. in 5% CO₂ in Dulbecco's-modified Eagle's medium (DMEM, Sigma) supplemented with 10% fetal bovine serum (FBS, Bioscicence), 1% glutamine, 1% Penicillin/Streptomycin (P/S) (GIBCO) until confluence. After dissociation in a 0.25% Trypsin-EDTA (GIBCO) solution and centrifugation, cells are re-suspended at a density of 1×10⁶ cells mL⁻¹.

Device preparation: Before cell seeding, the cell culture device, made of gelatin nanofibers and PEGDA, is sterilized under UV exposure for more than 30 min. A solution of fibronectin (FN) at 50 μg mL⁻¹ concentration (Sigma, France) in 0.1 M NaHCO₃ (pH=8) is used to coat the openings of the cell culture device at 37° C. for 30 min. The device is then placed in a culture dish and suspended in the cell culture medium; the said culture medium is loaded into the microchannels.

Off-ground cell culture using the device of the present invention: The cell suspension (200 μL) is introduced in the open areas of the cell culture system. After 30 min incubation, more culture medium is added into the Petri dish. Without any coating, both HeLa and NIH 3T3 can stick to nanofibers in 2 h. FIG. 9 shows the immunofluorescence images of NIH-3T3 and HeLa cells cultured using the device of the present invention and the merge of the three obtained images. For both of the cells, cytoskeleton and nuclear are respectively stained with Phalloidin-FITC and DAPI. Images obtained from Bright Field microscopy (BF) are also shown. Since PEGDA cannot absorb proteins, there is almost no cell fastened on the PEGDA grid, which demonstrated that cells can be patterned differently by easily changing the shape of the PEGDA grid.

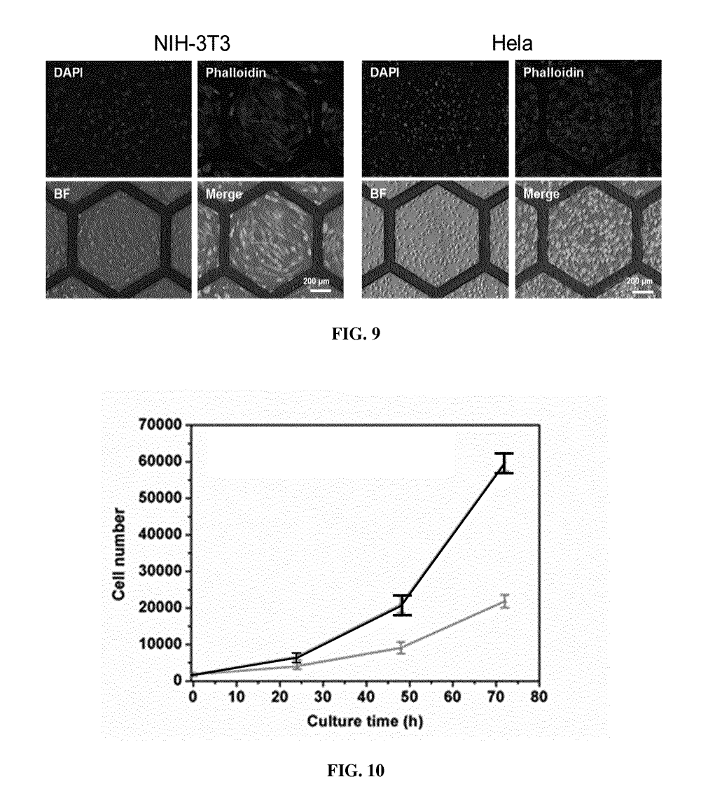

Then the inventors compare the doubling time of NIH-3T3 cells using the device of the present invention and a normal culture dish. Cells were digested down for counting cells number using a hemocytometer every day for 4 days, as shown in FIG. 10, the black line corresponding to the use of the present invention and the grey line corresponding to the use of a normal culture dish. After calculation, the doubling time and the growth rate of cells using the device of the present invention are respectively 15.01 h, shorter than using a normal culture dish (19.79 h), and 0.046, quicker than using a normal culture dish. (0.0350). It indicates that the proliferation rate of cells can be improved by using this kind of suspension culture. The device of the present invention allows a three dimensional nutrition supply environment for cells, rather than only up-side nutrition supply when using a normal culture dish.

### Example 5: hiPSCs Culture

Preparation of hiPSC: Human induced Pluripotent Stem Cells are prepared in complete E8 medium (life technology) with a vitronectin (life technology) coated culture dish at 37° C. with 5% CO2 supplementation. The medium is changed every day until cells grow to 70%˜80% confluences. Then, cells were harvested with a 0.5 mM EDTA DPBS solution.

Device preparation: To promote the adhesion of hiPSCs on gelatin fibers, the culture device (PEGDA grid and gelatin nanofibers) is coated with vitronectin diluted in PBS at a ratio of 1:500 at room temperature for 1 h. Then, the device is placed in a culture dish for cell seeding.

hiPSCs culture: hiPSCs at a cell density of 2×10⁵ in 50 μL E8 medium containing 10 μM ROCK inhibitor (Y-27632; Wako Chemicals) are plated on the surface of the cell culture device. The cell culture device is then placed in an incubator for 1 h hence allowing cell fastening. Then, 2 mL fresh E8 medium containing 10 μM ROCK inhibitor are gently added in the cell culture system. ROCK is a downstream effector protein which regulates both cell adhesion and migration by inhibiting depolymerisation of actin filaments and remodeling the actin cytoskeleton [WORTHYLAKE et al., J. Bio. Chem, 2003].

Therefore, inhibition of ROCK promotes cellular contraction and integrin-mediated adhesion and also prevents dissociation induced apoptosis and promotes the survival of embryonic stem cells and induced pluripotent stem cells [WATANABE et al., Nature Biotech., 2007]. After culturing for a given period, the culture medium is replaced by E8 medium without ROCK inhibitor. After 24 h, the formation of hiPSC aggregates is observed to determine the optimal culture conditions for the formation of dome-like aggregates.

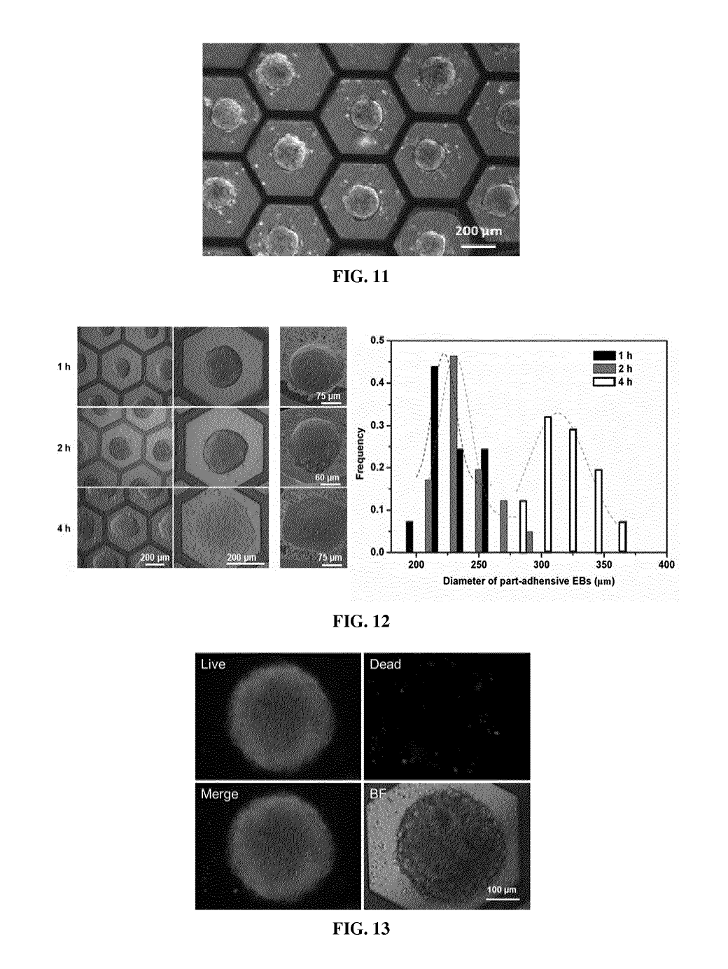

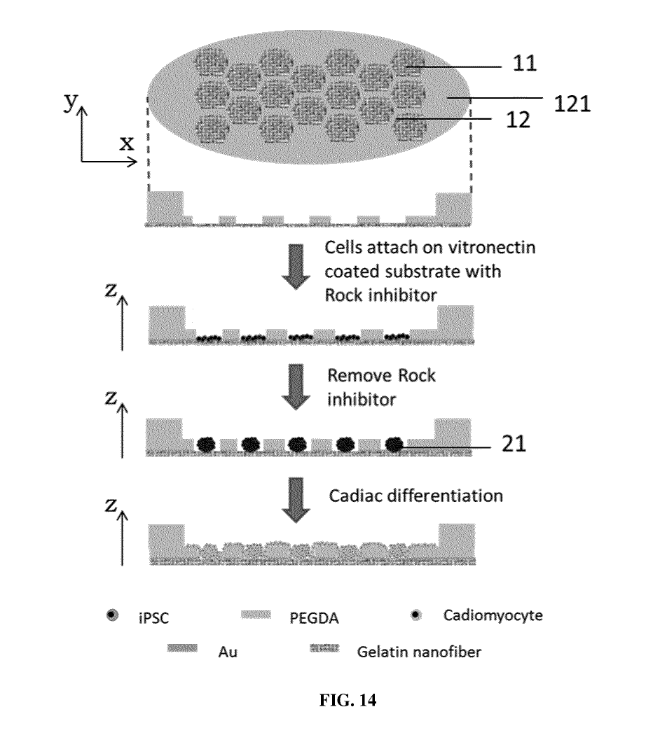

Shape control of hiPSCs colonies: iPSCs can tightly aggregate to form embryonic body like colonies in the center of the openings of the PEGDA grid on the gelatin nanofibers, no cells are found on the PEGDA grid, as shown in FIG. 11. Low concentration VN coated gelatin nanofibers are not sufficient to hold hiPSC colonies. By adding ROCK inhibitor during 1 h, hiPSC aggregates can stick to the monolayer gelatin nanofibers, showing dome-like hiPSC aggregates of diameter of 250 μm. As seen on images from the FIG. 12, with the increase of the duration of ROCK inhibitor treatment, the diameter of the aggregates increases while the dome height decreases, due to the enhanced adhesion to the nanofibers. After 2 h, the dome-like form of the aggregates is still preserved. Statistically, the average diameter of hiPSC colonies for 1 or 2 hours ROCK treatment is respectively about 220 to 230 μm. When the ROCK treatment is longer than 4 h, disk-like colonies are observed. To evaluate the viability of the hiPSC colonies 21 on monolayer of gelatin nanofibers, the dome-like aggregates are cultured for two more days and cells are stained with calcein AM and EthD-1 for live/dead assay. As shown in FIG. 13, almost all the cells of hiPSC colonies are alive and there are only few dead cells outside the colonies, indicting a high viability of cells on monolayer of gelatin nanofibers. This culture step is schematically describes in the first step of FIG. 14.

### Example 6: hiPSCs Differentiation to Cardiomyocytes

After 24 h generation of EBs (Embryoid Body), cardiac differentiation is conducted according to the protocol of [LIAN et al., Nature Protocol, 2013]. The process is schematically describes in the last step of FIG. 14. Briefly, the E8 culture medium is replaced by RPMI 1640/B27 culture medium without insulin but with 12 μM of CHIR99021 (GSK3 inhibitor). After 24 h incubation, the medium is replaced with RPMI 1640/B27 without CHIR99021 (day 1). After incubation of another 48 h, the culture medium is replaced with RPMI 1640/B27 without insulin but with 5 μM IWP2 (day 3).

After incubation of another 48 h, the medium is replaced with RPMI 1640/B27 without IWP2 (day 5). Then, the culture medium (RPMI/B27) is changed every three days. Generally, the contraction of the cells is observed during the period of 8 days to 12 days.

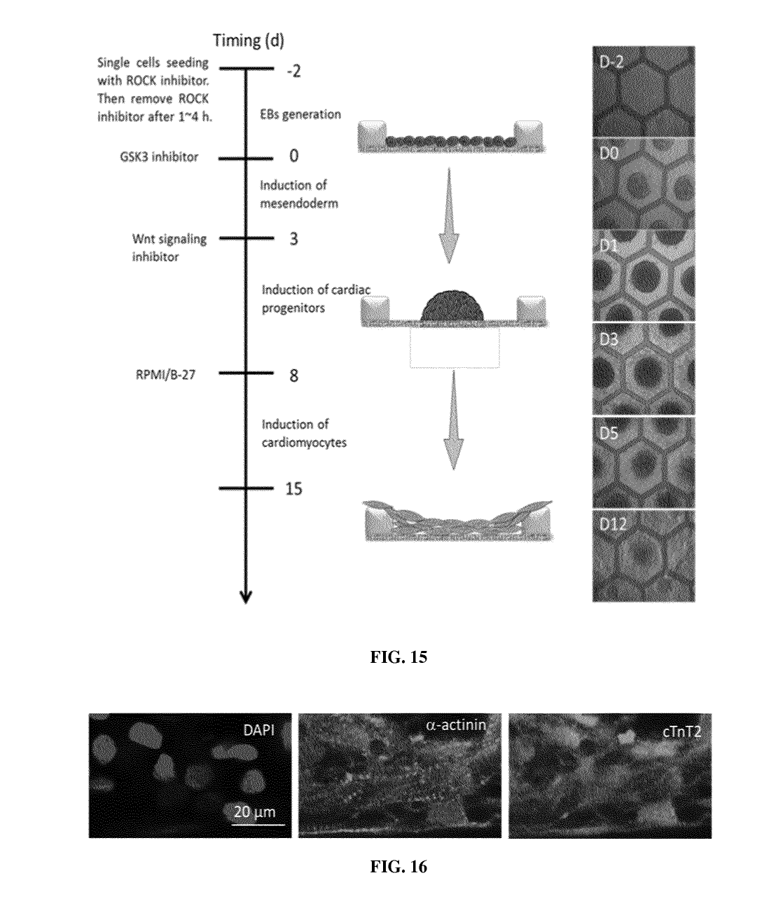

Cardiomyocytes differentiation on monolayer nanofibers: For cardiac differentiation, the use of dome-like hiPSC colonies might be advantageous, due to the fact that a close interaction with endodermal derivatives supports cardiomyogenic induction. Cardiac differentiation of hiPSC is achieved by using dome-like colonies without changing the monolayer of nanofibers. The different steps and SEM images of every steps are in FIG. 15. After 2 days culture, GSK3 inhibitor is introduced for mesendoderm (embryonic tissue layer) induction. At this stage, the colonies have almost the same shape. After another 3 days, Wnt (glycoproteins family) signaling inhibitor is added for the induction of cardiac progenitors. Progressively, the colonies become less compact. After another 5 days, spread colonies and beating of the cell clusters are observed. As shown in FIG. 16, in the end, the formation of sarcomeres is observed, indicating maturation of cardiomyocytes on monolayer of nanofibers.

### Example 7: hiPSCs Differentiation to Motor Neuron Progenitors

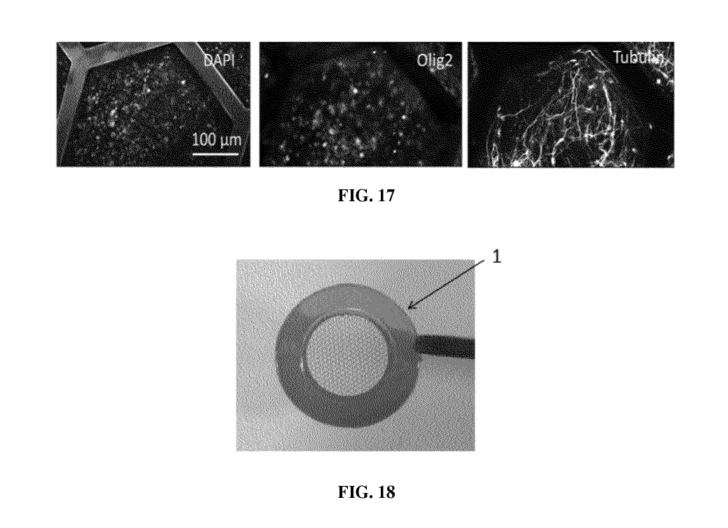

For neuroectoderm induction, iPSCs cultured on a monolayer of gelatin nanofibers are exposed to human neural induction medium consisting of DMEM/F12 supplemented with NEAA, Glutamax, LDN1931189, SB431542 and bFGF, according to the protocol of [SUN et al. Nature materials, 2014]. Upon 3 days of initial induction, N₂ medium is increased gradually every two days. Neuroectodermal cells can be obtained at Day 8. For motor neuron differentiation, cells are treated in the presence of retinoic acid and SHH for 8 days, with medium changed every two days. Motor neuron progenitors can be harvested at Day 16. As can be seen in FIG. 17, motor neuron progenitors can be obtained in the porosity of the monolayer of gelatin nanofibers.

1-15. (canceled)

16. An easy to handle and versatile cell culture device comprising:
a mesh comprising or made of a biocompatible polymer; and
a top grid comprising or made of a biocompatible polymer, laying over the said mesh;
wherein
the mesh is a monolayer of cross-linked nanofibers and has a specific surface ranging from 20% to 40%;
the top grid comprises a single grid and an array of openings separated by partitions having a width;
each opening of the top grid has the same geometric configuration; and
the top grid comprises a border surrounding the openings, the said border having a width at least two times greater than the width of the said partitions.

17. The cell culture device according to claim 16, wherein the said geometric configuration of the openings is a polygon.

18. The cell culture device according to claim 16, wherein each partition of the top grid separating the openings has the same cross-section.

19. The cell culture device according to claim 16, wherein the top grid has a border thicker than the partition of the top grid.

20. The cell culture device according to claim 16, further comprising a binding agent between the top grid and the mesh.

21. The cell culture device according to claim 16, wherein more than 50% of the pores of the said mesh have an area ranging from 0.01 to 20 μm².

22. The cell culture device according to claim 16, wherein the openings of the top grid have dimensions ranging from 200 to 1000 μm.

23. The cell culture device according to claim 16, wherein the nanofibers of the mesh comprise or are made of an hydrogel; or a doped hydrogel.

24. The cell culture device according to claim 16, wherein the nanofibers of the mesh comprise or are made of gelatin; or gelatin doped with carbon nanotubes.

25. The cell culture device according to claim 16, wherein the top grid comprises or is made of an hydrogel.

26. The cell culture device according to claim 16, wherein the top grid comprises or is made of poly (ethylene glycol) or poly (ethylene glycol) diacrylate.

27. The cell culture device according to claim 16, further comprising stem cells within the openings of the said top grid.

28. A cell culture system comprising:
at least one cell culture device according to claim 16; and
a culture medium.

29. The cell culture system according to claim 28, wherein the mesh and the top grid of the at least one cell culture device comprise or are made of hydrogels such that the at least one cell culture device may be suspended within the cell culture medium.

30. The cell culture system according to claim 28, further comprising an inlet port, an outlet port and a microchannel, wherein the said culture medium and the said at least one cell culture device are comprised within the microchannel.

31. A method for manufacturing a cell culture device comprising:
manufacturing a grid made from biocompatible polymer by soft-lithography;
depositing a nanofibers layer on the grid by electrospinning;
cross-linking the said nanofibers.

32. The method for manufacturing a cell culture device according to claim 31, further comprising depositing a binding agent on the grid by sputtering.

33. A method of stem cell growth or differentiation comprising the following steps:
providing a cell culture device according to claim 16; and
seeding at least one type of stem cells within the openings of the cell culture device in a medium optionally containing ROCK inhibitor.

34. The method of stem cell growth or differentiation according to claim 33, further comprising coating the cell culture device with glycoprotein such as vitronectin or fibronectin.

35. The method of stem cell growth or differentiation according to claim 33, further comprising removing ROCK inhibitor.
